(19) United States
(12) Plant Patent Application Publication
(10) Pub. No.: US 2002/0148025 P1
(43) Pub. Date: Oct. 10, 2002

(54) Kalanchoe plant named Parcival

(76) Inventor: Andre Smaal, Aalsmeer (NL)

Correspondence Address: ROCKEY, MILNAMOW & KATZ, LTD., Two Prudential Plaza, 180 North Stetson Avenue, Suite 4700, Chicago, IL, 60601, US

(21) Appl. No.: 09/782,631

(22) Filed: Feb. 13, 2001

(51) Int. Cl. A01H 5/00

(52) U.S. Cl. PLT/341

(57) ABSTRACT

A new and distinct Kalanchoe cultivar named Parcival is provided. This new cultivar is characterized by its light orange flowers, dark green foliage, compact growth habit and excellent basal branching.

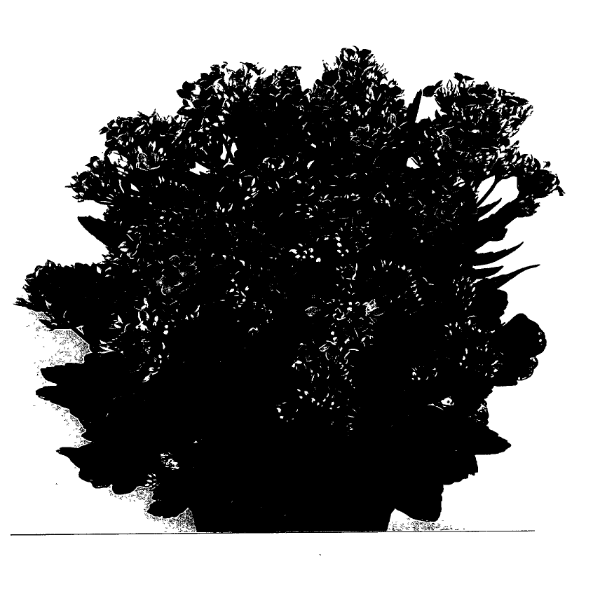

### BACKGROUND OF THE INVENTION

The present invention comprises a new and distinct Kalanchoe plant, botanically known as Kalanchoe blossfeldiana, and hereinafter referred to by the cultivar name Parcival.

The new cultivar was developed by the inventor through a controlled breeding program during August 1996 in The Netherlands. The female (seed) parent of Parcival was the proprietary Kalanchoe cultivar designated 93054, characterized by its orange flowers and good branching habit. The male (pollen) parent of Parcival was the proprietary Kalanchoe cultivar designated 94131-5, characterized by its orange flowers, dark green foliage and good branching habit. Parcival was discovered and selected as a flowering plant within the progeny of the stated cross by the inventor in June 1997.

Asexual reproduction of the new cultivar by terminal cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits orange flowers,

(b) forms dark green foliage,

(c) exhibits a good basal branching character and

(d) exhibits a compact upright mounded growth habit.

The new cultivar of the present invention can be compared to Petero (Not patented in the United States). In side by side comparisons, plants of the new cultivar differ from plants of Petero by the following characteristics:

1 The new cultivar is more compact.

2 The new cultivar exhibits flowers of a lighter orange color.

### BRIEF DESCRIPTION OF PHOTOGRAPH

The accompanying photograph shows as nearly true as it is reasonably possible to make the same in color illustrations of this type, typical flower and foliage characteristics of the new cultivar. The plants were grown in a greenhouse at West Chicago, Ill., U.S.A.

### DETAILED DESCRIPTION

The Parcival cultivar has not been observed under all possible environmental conditions to date. Accordingly, it is possible that the phenotype may vary somewhat with variations in the environment, such as temperature, light intensity, and day length.

The chart used in the identification of colors described herein is the R.H.S. Colour Chart of The Royal Horticultural Society, London, England. The color values were determined on Jan. 4, 2001 between 9:00 and 9:30 am under natural daylight. The plants used for the following observations and measurements were produced from cuttings taken from stock plants and grown in West Chicago, Ill. in 10 cm pots for 16 weeks. Plants were grown in a double polycarbonate covered greenhouse under conditions comparable to those used in commercial practice while utilizing a soilless growth medium and maintaining temperatures of approximately 82° F. during the day and approximately 66° F. during the night. Plants were exposed to three weeks of long days followed by 9 weeks of short days. Growth regulators were applied as follows: 3000 ppm B nine at days 17 and 28 and 1500 ppm day 42.

Classification:

Botanical.—Kalanchoe blossfeldiana, cultivar Parcival.

Parentage: Proprietary seedling 93054(seed)×Proprietary seedling 94131-5 (pollen)

Propagation:

Type cutting.—Terminal tip.

Time to initiate roots.—Approximately 7-14 day with shorter times in summer and longer times in winter.

Time to develop roots.—Approximately 21 days.

Root description.—Fibrous, branching.

Plant Description:

Habit of growth.—Moderately vigorous with good basal branching. A mature plant, at 16 weeks after the planting of an unrooted cutting, commonly measures approximately 12.9 cm in height and approximately 17 cm in width.

Branching habit.—Typically forms one main stem 10 cm in length and 9 mm in diameter and 9 to 10 lateral branches approximately 6 cm in length and 3 mm in diameter.

Plant form.—Upright and mounded.

Foliage.—Simple, opposite. Shape: Ovate with acute apex, cuneate base and crenate margin. Texture: Glabrous, succulent. Size of mature foliage: Approximately 8.2 cm in length and approximately 5 cm in width. Color of mature foliage: Upper surface closest to 137A, lower surface closest to 137C. Petiole length is approximately 1.5 cm, color is 144A.

Flower Description:

Flowering habit.—Freely flowering.

Natural flowering season.—Early January. Flowering time is dependent on temperature, light intensity and other growing conditions.

Inflorescence type.—Dichasiel cymes.

Flowers borne.—Above foliage.

Fragrance.—None.

Quantity of flowers.—Approximately 60 flowers on main stem and 45 flowers on each lateral branch.

Flower buds.—Shape: Elliptical. Length: Approximately 1.5 cm. Width: Approximately 4 mm. Color: 28D.

Perianth form.—Slaverform.

Flower size.—Approximately 1.7 cm in diameter, 3 mm in depth. Corolla tube is 1.2 cm long and 3 mm in diameter.

Petals.—Number: Four. Shape: Ovate with cuspidate apex, rounded base. Approximate size: 8 mm in length, 7 mm in width. Aspect: Slightly convex. Texture: Glabrous and velvety.

Sepals.—Number: Four. Shape: Lanceolate with acuminate apex, entire margin. Texture: Smooth. Color: Upper and lower surfaces between 144C and 144D.

Mature flower color.—Upper surface of petals is closest to 26B with streaks of 34C, fading to 32C then to 35A at senescence; lower surface of petals is closest to 28D.

Peduncle.—Length: Approximately 3.3 cm in length and 4 mm in diameter.

Pedicel length.—Approximately 5 mm.

Reproductive organs.—Androecium: Stamen number: 8 in two groups of four each with one group shorter than the other. Anther shape is oblong and flat. Anther length: 1 mm. Pollen color: 2B. Gynoecium: Stigma shape: Flat. Stigma appearance: Crystalline, colorless and transparent. Style length: 2 mm. Style color: 151 C. Ovary : 4 celled, 7mm in length, 144A.

Seed development: Seed production has not been observed.

Disease resistance: Resistance to pathogens has not been observed.

I claim:

1. A new and distinct cultivar of Kalanchoe named Parcival substantially as herein shown and described, which:
(a) exhibits orange flowers,
(b) forms dark green foliage,
(c) exhibits a good basal branching character and
(d) exhibits a compact upright mounded growth habit.
TABLE A

| CHARACTERISTIC | PARCIVAL | PETERO |
|---|---|---|
| PLANT HEIGHT-FROM SOIL | 13.2 CM | 20.5 |
| TO TOP OF FLOWERS | | |
| MAIN COLOR OF MATURE | 26 C | 29 A |
| PETALS | | |
